(19) United States
(12) Patent Application Publication
(10) Pub. No.: US 2026/0272695 A1
(43) Pub. Date: Sep. 17, 2026

(54) THERMALLY RECONFIGURABLE VITRIMER-BASED ORTHOPEDIC CAST FOR DISTAL RADIUS FRACTURE IMMOBILIZATION

(71) Applicant: Alfaisal University, RIYADH (SA)

(72) Inventors: Layan Albraik, Riyadh (SA); Areej Alwabil, RIYADH (SA); Leen Albraik, RIYADH (SA); Thamer Alraiyes, RIYADH (SA); Yousef Algoos, RIYADH (SA); Wejdan Munawir Alamri, RIYADH (SA)

(73) Assignee: Alfaisal University, RIYADH (SA)

(21) Appl. No.: 19/564,163

(22) Filed: Mar. 12, 2026

Related U.S. Application Data
(60) Provisional application No. 63/772,455, filed on Mar. 15, 2025.

(51) Int. Cl. A61F 5/058; B33Y 50/00; B33Y 80/00

(52) U.S. Cl. CPC A61F 5/05866; B33Y 50/00; B33Y 80/00

(57) ABSTRACT

An orthopedic cast made of a vitrimer polymer lattice shell additively manufactured from anatomical scan data. The cast provides ventilation, structural immobilization, and thermal reshaping capability while maintaining mechanical integrity. A cast system for adult distal radius fractures is disclosed, a vitrimer-based polymer lattice shell custom-generated through anatomical modeling and additive manufacturing. A patient-specific immobilization device with modular segments and structural zones tailored for the distal forearm and wrist region. Using 3D anatomical input and a proprietary digital workflow, the system calculates fracture-appropriate cast geometry, including variable wall thickness and ventilation cell size. The cast design accommodates fracture type, swelling potential, and ergonomic function while permitting surgical site access.

### CROSS-REFERENCE TO RELATED APPLICATIONS

This application claims priority to U.S. Provisional Patent Application No. 63/772,455, filed Mar. 15, 2025, the entirety of which is incorporated herein by reference.

### FIELD OF THE INVENTION

The present invention relates to orthopedic immobilization devices and, more particularly, to a patient-specific distal radius fracture cast formed from a vitrimer polymer using additive manufacturing and 3D anatomical modeling.

### BACKGROUND OF THE INVENTION

The treatment of distal radius fractures in adults, particularly those involving complex or displaced patterns, commonly requires temporary or extended immobilization using rigid orthopedic casts. Traditional cast materials, including plaster of Paris and fiberglass composites, although effective at restricting motion, suffer from several critical limitations: non-breathability, excessive weight, water retention, and an inability to accommodate dynamic anatomical changes due to swelling, surgical access, or follow-up care. Moreover, these materials necessitate full cast removal for any visual inspection, often disrupting healing or requiring redundant procedures.

Recent explorations into 3D-printed cast solutions using lattice designs have offered alternatives with improved ventilation and aesthetic customization. However, these prior solutions are typically constructed from thermoplastics such as PLA or PETG, which lack the thermal and mechanical flexibility needed for clinical adjustments. Furthermore, these systems often rely on rigid template-based modeling with minimal adaptation to patient-specific anatomical and biomechanical variation.

Distal radius fractures are among the most common orthopedic injuries requiring immobilization. Conventional casts made from plaster or fiberglass are heavy, poorly ventilated, and not adaptable to patient anatomy. Existing 3D printed casts use thermoplastics that cannot be reshaped without structural degradation. There remains a need for an anatomically customized, ventilated, structurally stable cast capable of reshaping without loss of mechanical integrity.

### SUMMARY OF THE INVENTION

The invention provides an orthopedic cast comprising a lattice shell fabricated from a vitrimer polymer composition. The cast is generated from anatomical scan data and additively manufactured to conform to patient anatomy. The vitrimer composition enables thermal reshaping while maintaining structural integrity, allowing patient-specific fitting and adjustment.

In one embodiment, a customized orthopedic cast system specifically designed for the immobilization of adult distal radius fractures is disclosed. The said system comprises of: (a) a dynamic vitrimer-based material composition designed for thermal reconfigurability and orthopedic-grade strength, and (b) a digital fabrication method that produces cast geometries based on anatomical input, fracture classification, and clinical access needs.

The cast is generated through a software pipeline that interprets 3D anatomical scan data of the patient's forearm, identifies bony landmarks, and assigns lattice densities, strut dimensions, and shell segment outlines based on curvature and loading analysis. The generated cast includes ventilation apertures, an anterior access window for surgical site exposure, and modular lateral shells that can be opened and re-secured using integrated fasteners or latching segments.

The vitrimer material used in the cast enables thermal modification after printing, permitting clinicians to reshape portions of the cast under controlled heat (typically 120-160° C.) to accommodate changes in limb volume or ensure improved ergonomic fit. Unlike thermoplastics, vitrimer exhibits no degradation in mechanical integrity during bond exchange, providing an ideal material platform for reusable, patient-adapted immobilization.

The invention claims protection over both the lattice cast geometry and the vitrimer formulation, as well as the method of data acquisition, digital model generation, and fabrication. The system supports efficient on-site or near-site production, reducing supply chain dependency and allowing same-day cast delivery customized to individual patient anatomy and fracture type.

### BRIEF DESCRIPTION OF THE DRAWINGS

Example embodiments are illustrated by way of example and not limitation in the figures of the accompanying drawings, in which like references indicate similar elements and in which:

FIG. 1 illustrates dorsal view of the orthopedic cast.

FIG. 2 illustrates palmar view of the orthopedic cast.

FIG. 3 shows the dorsal and palmar view of the orthopedic cast.

FIG. 4 illustrates assembly of cast segments with screws.

Other features of the present embodiments will be apparent from the accompanying drawings and from the detailed description that follows.

### DETAILED DESCRIPTION

This disclosure illustrates and enables system, method and formation of orthopedic cast (an immobilization device) made of advanced polymeric material by additive manufacturing. Specifically, it pertains to the design, formulation, and digital fabrication of a forearm cast for adult distal radius fracture treatment. The cast structure features joint-specific geometry, lattice shell architecture, and thermally reconfigurable vitrimeric polymer composition, integrated into a single system optimized for patient-specific digital workflows. An orthopedic cast made of a vitrimer polymer lattice shell additively manufactured from using patient specific anatomical scan data. The orthopedic cast provides ventilation, structural immobilization, and thermal reshaping capability while maintaining mechanical integrity. An orthopedic cast system for adult distal radius fractures is disclosed, a vitrimer-based polymer lattice shell custom-generated through anatomical modeling and additive manufacturing. A patient-specific immobilization device with modular segments and structural zones tailored for the distal forearm and wrist region. Using 3D anatomical input and a proprietary digital workflow, the system calculates fracture-appropriate cast geometry, including variable wall thickness and ventilation cell size. The cast design accommodates fracture type, swelling potential, and ergonomic function while permitting surgical site access.

The orthopedic cast comprises dorsal and palmar shell segments fabricated using additive manufacturing from a vitrimer polymer composition. The vitrimer composition includes an epoxy resin, crosslinking agent, and catalyst enabling dynamic covalent bond exchange. Anatomical scan data is used to generate patient-specific geometry with spatially varying lattice density. The cast provides ventilation, structural support, and thermal reshaping capability without degradation of mechanical properties.

Vitrimer Material Formulation: The present invention utilizes a vitrimeric polymer blend engineered for orthopedic casting applications. Vitrimers, a subclass of polymer networks characterized by dynamic covalent bond exchange, combine the thermal stability of thermosets with the reprocessability of thermoplastics. The Lalbraikast formulation employs an epoxy-based vitrimer system catalyzed by either zinc acetate or organic Lewis base compounds that mediate transesterification reactions.

The formulation comprises of: Base Resin: Epoxy diglycidyl ether of bisphenol A (DGEBA), serving as the primary network former. In one example the Crosslinking Agent: A diacid or anhydride-based linker with terminal carboxylic acid groups (e.g., sebacic acid). In one example the Catalyst: Zinc-based catalyst (e.g., zinc acetate) in a concentration of 2-5 wt %, enabling associative bond exchange. In one example the Fillers: Nano-silica or carbon fiber additives (1-3 wt %) may be incorporated to enhance modulus, fatigue resistance, and print fidelity.

The vitrimer transitions to a reconfigurable state at 120-160° C., allowing the cast to be locally reshaped during or after clinical fitting. The material maintains structural rigidity below its topology freezing transition temperature (Tv), ensuring effective immobilization at room and body temperatures.

The polymer is fabricated into filament or pellet form and is compatible with commercially available FDM (fused deposition modeling) or pellet-extrusion 3D printers. The final mechanical properties are tuned to match or exceed those of conventional fiberglass casting material, with added benefits of ventilation, lightness, and reshaping potential.

Anatomical Input Acquisition: The generation of a patient-specific Lalbraikast begins with anatomical data capture. The system supports the following scanning modalities:
Handheld 3D Structured Light Scanners (e.g., Artec Eva, Einscan HX)
Mobile LiDAR-Based Devices (e.g., iPad Pro with LiDAR)
Photogrammetry Reconstruction using a calibrated series of 2D images

Scanned data is processed using mesh-cleaning algorithms that remove artifacts, align the limb to a neutral axis, and identify bony landmarks of the distal radius, ulna, and carpal articulation. The digital model distinguishes dorsal and palmar surfaces and calculates the region requiring immobilization based on fracture classification. The cast system is designed exclusively for adult patients and considers anthropometric variations through database comparisons. User input may include patient height, weight, activity level, and dominant hand, allowing for optimized lattice density and cast wall stiffness.

Lattice Architecture and Design Parameters: The cast structure is built from a hexagonally-tiled lattice array projected along the curvature of the forearm and wrist. The lattice pattern is not uniform but varies based on anatomical location:
High-stress zones (e.g., dorsal wrist, distal radial ridge): Dense lattice nodes, 2-3 mm strut spacing, 4-5 mm strut thickness
Low-load zones (e.g., mid-forearm, soft tissue interface): Larger lattice spacing (5-8 mm), reduced strut thickness (2-3 mm)
Inspection zone: Anterior window of 25-40 mm, bordered by reinforced rim structure to prevent local stress concentration

The cast comprises two modular halves:
Palmar Shell: Contours from the metacarpal heads to the mid-forearm; includes thumb channel and radial support structure.
Dorsal Shell: Provides posterior reinforcement and overlap with the palmar segment; includes closure notches and side apertures. Closure is achieved through snap-fit joints or bio-compatible strap interfaces along the lateral margins. The cast may be assembled around the patient's limb or pre-formed and adjusted thermally at the point of care.

Wall Thickness and Structural Optimization: Wall thickness in the Lalbraikast is not constant. Instead, it is determined through a parametric algorithm that considers:
Curvature Gradient: Steeper curves (e.g., radial head, wrist crease) receive additional material layering
Predicted Load Zones: Forearm orientation during typical activity is used to model compressive and torsional forces
Patient Mass Index: Heavier individuals receive increased cast thickness to distribute stress

In one embodiment a standard adult configuration:
Distal radius zone: 4.5-5.0 mm thickness
Mid-forearm: 3.5-4.0 mm
Strap zones and closure interfaces: 5.5-6.0 mm

This ensures rigidity where immobilization is critical, while reducing unnecessary bulk where only structural support is needed. Finite element analysis (FEA) has been used to validate the pressure distribution and mechanical yield of the lattice under physiological loading conditions, confirming a safety factor >2 for daily stress scenarios.

Manufacturing and Application Workflow: Once the design is finalized, the STL (standard tessellation language) file is exported to a slicing program. Recommended printing parameters for FDM printing with vitrimer filament are:
Nozzle temperature: 220-230° C.
Bed temperature: 70-90° C.
Print speed: 40-60 mm/s
Layer height: 0.2-0.3 mm
Cooling fan: Off or low setting to ensure layer adhesion

For complex lattice angles, support structures may be generated with breakaway or soluble materials. The cast is printed in two separate halves and assembled at the clinic. A post-print thermal calibration step allows clinicians to heat-adjust the cast along the inner wrist arch for a snug yet comfortable fit.

Clinical application time is estimated as follows:
Scan to file generation: <30 minutes
Printing: 2.5-3.5 hours (depending on printer and resolution)
Thermal fitting and assembly: <20 minutes
   The entire process is thus suitable for same-day application in orthopedic clinics or hospital settings equipped with on-site fabrication labs.

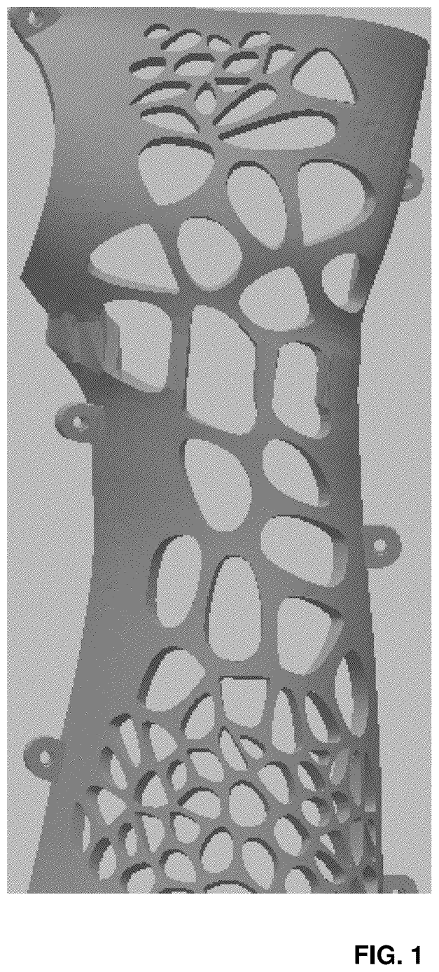

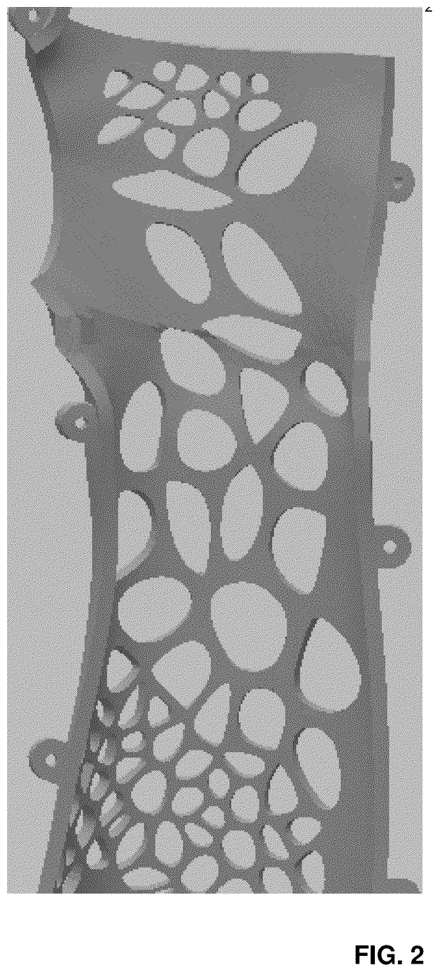

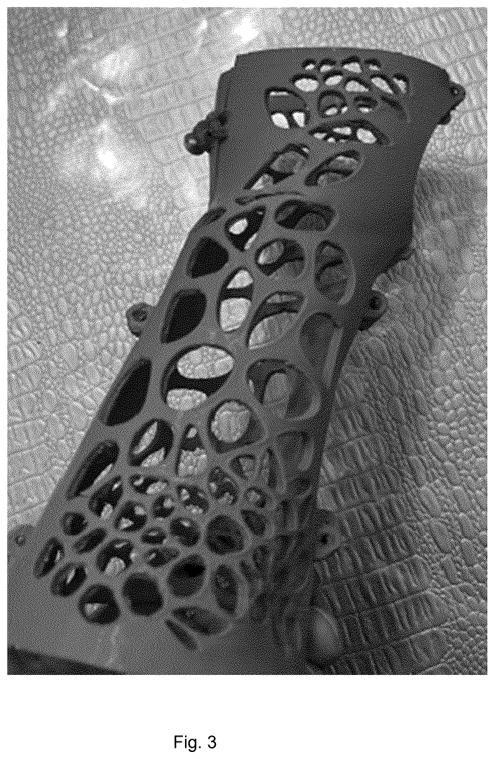

FIG. 1 shows the dorsal view of the cast for patient specific requirements. It also shows the lattice design. FIG. 2 shows the palmar view of the orthopedic cast that matched the outer edges of the dorsal cast. FIG. 3 shows the two sides of the cast aligned with each other before it is installed on the specific patient.

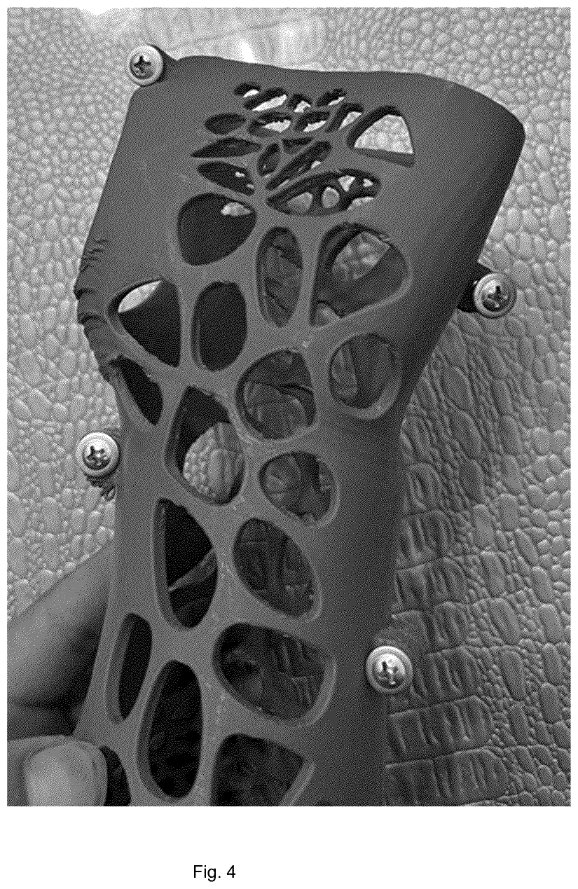

FIG. 4 shows, in one embodiment, the assembled cast that is customized and ready to be put on the hand of the specific patient and due to screw assembly the ease of removal and and flexibility. The doctors have enough space to do procedures and x-ray can be done by just removing the screws and not disturb the entire cast. The lattice design provides breathing room and the skin is not irritated.

Industrial Applicability: The orthopedic cast system is highly applicable within clinical, surgical, emergency, and rehabilitation contexts for the treatment of distal radius fractures in adult patients. The system is compatible with existing digital workflows in orthopedic clinics and trauma centers and can be deployed with commercially available scanning and 3D printing equipment. Its ability to integrate patient-specific anatomy, mechanical stability, breathability, and post-fabrication thermal adaptability make it especially well-suited for:
Hospitals with in-house or partner additive manufacturing capabilities.
Orthopedic practices aiming to provide same-day, custom immobilization.
Military and sports medicine clinics requiring durable yet lightweight immobilization with wound access.
Regions with unreliable supply chains where on-demand local fabrication is preferred.

The system reduces overall material waste compared to traditional cast fabrication and enables efficient turnaround, personalized treatment, and patient satisfaction. It further offers a platform for digital archiving, follow-up fitting, and adaptation to evolving injury needs—all of which are key for modern orthopedic care.

What is claimed is:

1. An orthopedic cast for immobilization of a distal radius fracture in an adult patient, comprising:
a dorsal shell segment and a palmar shell segment configured to surround at least a portion of a forearm and wrist of the adult patient;
each segment comprising a lattice structure formed from an interconnected struts defining a ventilation opening;
wherein the lattice structure is formed from a vitrimer polymer composition comprising an epoxy resin, a crosslinking agent, and a catalyst enabling associative bond exchange.

2. The orthopedic cast of claim 1, wherein the epoxy resin comprises diglycidyl ether of bisphenol A.

3. The orthopedic cast of claim 1, wherein the catalyst comprises zinc acetate.

4. The orthopedic cast of claim 1, wherein the vitrimer polymer is thermally reconfigurable.

5. The orthopedic cast of claim 1, wherein the lattice spacing ranges between 2 mm and 8 mm.

6. The orthopedic cast of claim 1, wherein the strut thickness ranges between 2 mm and 5 mm.

7. The orthopedic cast of claim 1, comprising a snap-fit closure features.

8. The orthopedic cast of claim 1, comprising an inspection opening.

9. The orthopedic cast of claim 1, comprising a reinforcing filler.

10. The orthopedic cast of claim 9, wherein the reinforcing filler comprises nano-silica or carbon fiber.

11. The orthopedic cast of claim 1, capable of reshaping at 120° C.-160° C.

12. A method of generating the orthopedic cast, comprising the steps of:
(a) acquiring a 3D anatomical scan of the patient's forearm and wrist;
(b) processing the scan data to extract bone landmarks and limb curvature;
(c) generating a digital lattice shell geometry with joint-specific structural reinforcements and ventilation;
(d) exporting the cast model as an STL file for additive manufacturing; and
(e) printing the cast using vitrimer filament and assembling the segments post-print.

13. The method of claim 12, wherein localized thermal adjustment is applied to the cast surface post-print to improve fit and pressure distribution, without compromising mechanical function.

14. An orthopedic cast system designed for an adult distal radius fracture, comprising:
a lattice-structured shell produced from a vitrimer polymer; said shell generated through additive manufacturing; wherein an orthopedic cast geometry conforms to a patient-specific anatomy and provides ventilation, structural support, and anterior access.

15. The system of claim 1, wherein the vitrimer polymer comprises an epoxy-based resin crosslinked through dynamic covalent chemistry, enabling reshaping upon application of localized heat between 120° C. and 160° C.

16. The system of claim 1, wherein the cast geometry includes a parametric lattice with variable wall thickness ranging from 3.5 mm to 6.0 mm, wherein the computation is based on an anatomical curvature and a predicted stress distribution, wherein the orthopedic cast covers a dorsal and a palmar segment, joined through a lateral snap-fit or strapping closure mechanism allowing post-fabrication assembly on a patient.
